(19) United States
(12) Patent Application Publication
(10) Pub. No.: US 2017/0329927 A1
(43) Pub. Date: Nov. 16, 2017

(54) SYSTEM AND METHOD FOR ANALYZING AIRWAY-PULMONARY RESPONSE USING COMPUTATIONAL FLUID DYNAMICS TO DIAGNOSE AND MONITORING POTENTIAL HEALTH ANOMALIES

(71) Applicant: InFluidS LLC, Long Beach, CA (US)

(72) Inventors: Shahab Taherian, Los Angeles, CA (US); Jeremy Bonifacio, Huntington Beach, CA (US); Hamid Rahai, Long Beach, CA (US); Thomas Waddington, Huntington Beach, CA (US); Bernardo Gomez, El Segundo, CA (US)

(21) Appl. No.: 15/431,103

(22) Filed: Feb. 13, 2017

Related U.S. Application Data
(60) Provisional application No. 62/335,018, filed on May 11, 2016.

(51) Int. Cl. G06F 19/00; A61B 5/087; A61B 5/00; A61B 5/055; A61B 6/00; A61B 8/08; G06T 7/00

(52) U.S. Cl. CPC G06F 19/3437; G06T 7/0012; A61B 5/087; A61B 5/4818; A61B 6/50; A61B 6/5217; A61B 8/08; A61B 8/5223; A61B 5/055; G06T 2207/10028; G06T 2207/10081; G06T 2207/10088; G06T 2207/10104; G06T 2207/10108; G06T 2207/10132; G06T 2207/30061

(57) ABSTRACT

A CFD-based diagnostic system can be used as a non-invasive diagnostic and monitoring tool for ECAC, central airway obstruction diseases, OSA and airway stenosis. The process is expected to reduce the time of diagnosis, number of tests, and hospitalization time.

### CROSS-REFERENCES TO RELATED APPLICATIONS

This application claims priority from U.S. application Ser. No. 62/335,018, filed May 11, 2016 incorporated by reference in its entirety.

### BACKGROUND

The invention focuses on expiratory central air way collapse (ECAC); ECAC is the narrowing of the airways during expiration, which is divided into Tracheomalacia (TM)/Tracheobronchomalacia (TBM) and Excessive Dynamic Airway collapse (EDAC). The invention non-invasively monitor/diagnose ECAC, and/or differentiate between TBM and EDAC diseases using computational fluid dynamics (CFD) and medical imaging modalities (specifically dynamic computerized tomography (CT) scans), based on the degree of air flow obstruction, flow structure, pressure drop and local wall shear stresses.

The following will discuss the incident/prevalence, classification, possible causes, traditional diagnosis modalities/treatments of EDAC/TBM; the invention and results for non-invasive evaluation of these diseases is presented.

Incident and Prevalence

Infections, inflammatory disorders, malignancy and extrinsic compression from adjacent structures can result in tracheal disorders. Narrowing or stenosis, an upper airway obstruction, is the manifestation of these disorders. Compared to lower airway obstructions, tracheal obstructions can be life threatening, since there are no collateral ventilations that can distribute the air to other airway branches.

One large general study suggests that acquired TBM was reported to be in 4.5% of bronchoscopies performed for evaluation of respiratory disease and patients with a history of chronic bronchitis. There are several studies proposing a strong correlation between prevalence and incidents of chronic obstructive pulmonary disease (COPD) and EDAC/TBM. Review of a general population study implies that TBM incidents lie between 5-10% for patients suffering from asthma and COPD. In another study, which considered a cohort of 202 asthmatic patients, TBM was observed among 9.4% and EDAC among 30.7% of patients.

The incidence of EDAC and TBM are highly dependent on the narrowing airway lumen percentage criteria (>50% reduction and in some cases >80% reduction), and it is reported to be between 4-22% in patients with COPD and/or asthma.

COPD is the fourth and fifth leading causes of death in the United States and worldwide respectively, and the numbers are growing rapidly. COPD is diagnosed via the pulmonary function test (PFT), and in the United States, from 1999-2011, 12.7 million adults have been diagnosed with COPD. The National Heart, Lung, and Blood Institute's annual health expenditures projection (direct healthcare, indirect mortality and morbidity) was estimated at $49.9 billion in 2010. Cigarette smoking is the most important risk factor for COPD.

Although PFT is generally used for diagnosing COPD, a significant correlation has not been identified in determining the degree of collapse in ECAC with PFT results, causing false positive diagnosis of COPD for patients with just EDAC or not accounting for EDAC/TBM all together in patients with COPD or asthma.

The prevalence and incidence of EDAC and TBM in patients with COPD and asthma, their associated cost and hospitalization, highlights the importance of an innovative diagnosis/monitoring system that is not solely dependent on PFTs.

Disease Classification

Although EDAC is often mentioned interchangeably and incidentally with TBM, there are morphological and pathophysiological differences between these diseases.

TM and TBM are characterized by weakness of the cartilaginous structure in the trachea and can extend to one or more main bronchi respectively. EDAC is the collapse of the posterior membrane wall during exhalation or coughing, and it is unrelated to cartilage function and collapse.

TBM and EDAC can be classified into five domains: functional impairment, morphology, origin, length and location abnormality, and severity of airway collapse. However, classifications are not generally obvious and additional parameters, such as flow behavior, can significantly help with the assessment, pre-operative planning and post-therapy monitoring of these diseases.

The morphological structure is used for classification of TBM and EDAC. The anteroposterior narrowed wall is called the “Crescent Type” (the most common type), and the “saber-sheath type” is the lateral wall narrowing for the morphological classification of TBM. However, to see if these morphological changes cause flow limiting, the decreased pressure must be measured along the collapsed segment. With the proposed system we can non-invasively measure the percentage pressure drop along the flow limiting segment, which is demonstrated in the related work section of this invention.

ECAC is generally associated with COPD in patients with a history of smoking, thus concluding that smoking and chronic inflammation are contributing factors to TBM and EDAC; however the cause of acquired TBM in many patients is not obvious, thus labeling TBM patients as smokers may lead to insurance companies denying claims for non-smokers benefits on the grounds of “false indication of non-smoking status”.

There have been attempts in literature to standardize the differences between EDAC and TBM with regards to the five domains mentioned above, however the effect of air flow on the airway structure and its relationship to these diseases, in physiological and pathological states, have not been investigated.

The separation of TBM and EDAC as different disease entities is relatively new, and it is essential for treatment options and procedures.

TBM and EDAC-Associated Diseases

There are various diseases that can be associated to ECAC. Physiological studies demonstrate that collapsed airways in EDAC patients might be the consequence of COPD, resulting from decreased elastic recoil, small airway inflammation and atrophy of elastic fiber, asthma, bronchiectasis and bronchiolitis due to small airway inflammation, and obesity due to positive pleural pressure.

Tracheomalacia is generally referred to as weakness or loss of cartilage integrity, causing softer airways likely to collapse due to congenital diseases or acquired through chest trauma, chronic recurrent airway infections, and cancer or a tracheostomy tube.

TBM and EDAC Diagnosis and Treatments

There are different diagnosis modalities that can be used for the diagnosis of TBM/EDAC, which can be categorized into invasive and non-invasive diagnosis. The gold standard for dynamic tracheal collapse visualization is by bronchoscopy, which is an invasive procedure. CT scans can be used as a non-invasive tool for diagnosis of ECAC. CT-scans provide additional information, on vasculature, masses/tumors or parenchymal changes near the trachea, to understand the causes of ECAC. However the static inspiratory or expiratory phase cannot truly indicate the degree of airway collapse, and thus making dynamic CT-scans a superior choice. Generally CT-scans are used for pre- and post-operation (e.g. stent) assessment, in order to monitor possible complications such as migration, mucus obstruction or choke point migration.

In order to keep the airway open for EDAC and TBM, the treatments can range from surgical interventions to stent placement, medication and using continuous positive airway pressure (CPAP)/positive expiratory pressure (PEP) machines. Surgical interventions, such as tracheostomy (to maintain a stable airway structure), tracheoplasty/splinting (to reshape the airway structure), among others, are invasive and irreversible surgical procedures that canhave complications. Accurate assessments of the diseases are essential, before performing these interventions which highlight the importance of this invention.

Stents can provide support for the collapsing airways, metallic and silicon stents are used for these types of disease, with the former being the primary choice due to fewer complications. If the airway opening after the stent is compromised, other interventions might be necessary; making the system purposed here for a non-invasive assessment of pressure and flow for pre- and post-operations a valuable tool.

As a foreign body, stents generally may cause disturbances in the clearance of mucosa, resulting in plugging with a tendency to initiate local infection sites. In order to monitor the stent's possible adverse effects, a follow-up bronchoscopy or dynamic CT have been used. However the effect of location of stent placement on airflow has not systematically been considered in the clinical setting; this is demonstrated in the example section, which highlights the application of invention regarding pre/post intervention and choke point migration monitoring to assess if further intervention is required. This CFD-based diagnostic system can be used as a non-invasive diagnostic tool for pulmonary hypertension (PH), pulmonary embolism (PE), central airway obstruction diseases and airway stenosis.

From a clinical stand point of view since surgical intervention and stenting are invasive in nature, pharmaceutical medical treatments are the initial efforts to treat the underlining causes of EDAC. Bronchodilators, steroids and antibiotics can treat chronic bronchitis as the underlining cause of EDAC in some cases; however medical pharmaceutical treatments are not always effective and may have adverse effects.

Non-invasive treatments such as PEP and CPAP therapies are used, via a nose/mouth piece, to introduce positive pressure into the respiratory system in order keep the airway open; the non-invasive nature of this system is highly effective in cough, and secretion management in ECAC patients and also for patients with obstructive sleep apnea (OSA).

Finding an accurate positive pressure in PEP and CPAP therapies is labor intensive, cumbersome and requires incremental pressure adjustment from 0-20 cm H2O to evaluate the degree of airway narrowing and to keep the airways open. In order to find this value, bronchoscopy with noninvasive positive pressure ventilation (NIPPV) is performed. The proposed system (CT-CFD based monitoring system) can predict the pressure needed to keep the airway open in PEP and CPAP, eliminating the need for incremental pressure adjustments and bronchoscopy.

### SUMMARY OF THE INVENTION

The objective of the invention is a non-intrusive system that incorporates the medical images and CFD analysis to monitor and diagnose diseases to reduce the time and cost of diagnosis. One embodiment of the present invention is the biomarkers provided to diagnose and monitor ECAC diseases.

Another object of the invention is to investigate the relationship between the shape of a collapsed airway and the characteristic of the flow for differentiation between various ECAC diseases. This can be accomplished based on the flow characteristics (e.g. Coanda effect for EDAC), length of recirculating zones, eddy formations and the strength of the secondary flows (i.e. Dean flow), airway resistance caused by different morphological structure.

Spirometry (one of the PFT measurements) results from stent interventions or tracheoplasty do not necessarily show the percentage of symptom improvement before and after interventions; it might suggest improvements in coughing or pulmonary mechanics, however it does not show the degree of airflow improvement measured by FEV1%. By using the present invention, one can predict the percentage airflow improvement.

Another objective of this invention is to accurately predict the optimal location of the stent placement and interventions.

The present invention also concerns a method for assessing/diagnosing obstructive sleep apnea (OSA) using a CFD-CT based system, based on fluid characteristics, velocity magnitude and pressures gradients.

### BRIEF DESCRIPTION OF THE DRAWINGS

FIG. 1 is a flow chart for carrying out the general process of the present invention.

FIG. 2 illustrate pre-stent and post-stent CFD analysis for the patient of Table 1;

FIG. 3 is a graph showing inspiratory pressure-expiratory pressure (P-P) for pre-stent and post-stent; and

FIG. 4 is a graph showing Transverse sectional area type Cp and velocity magnitude comparison in ECAC diseases.

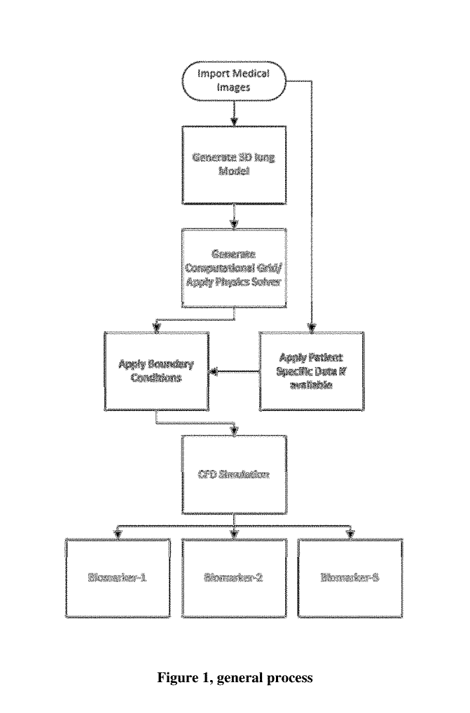

### DETAILED DESCRIPTION OF THE PREFERRED EMBODIMENTS

In step 1, the medical images are imported from CT, inspiratory or expiratory phase CT, dynamic CT, Magnetic Resonance Imaging (MRI), Cine MRI technique (dynamic), single-photon emission CT (SPECT)/positron emission tomography (PET) or ultrasound, while different segmentation algorithms in step 2 are applied to isolate, extract and generate the 3-D model from the medical images. The 3-D model is prepared for simulation in step 3, and patient specific boundary conditions are assigned in step 4 for the CFD simulation (step 5). After the simulation is completed the results are illustrated in step 6. If future optimization is required, the boundary conditions are adjusted and the process is repeated beginning from step 4.

Medical Imaging is performed and preferred but not limited to the patient being awake.

The conversion of medical images to 3-D models is the next step. From the patient specific 3-D model, inlets and outlets modification are applied for any specific anatomical structures changes that may or may not be present in the patient images. The advantage of changing the model at this point is to allow for a more accurate flow representation if any structure is absent in the image but is present in the patient. In this step, other model corrections can be made to the structure to simulate post interventions.

Once the 3-D model is generated, it is prepared for CFD analysis and converted to computational models. This converted model can be developed using any of the art, which may include different meshing techniques; preferably, but not limited to polyhedral and prism layer meshes.

When the 3-D meshed model is ready, proper physics models/equations are applied. The Reynolds-Averaged Navier Stokes equations (using but not limited to the shear-stress transport (SST) K-ω turbulence or other two equation models) or detached eddy simulation (DES) model solver is used to investigate the flow characteristics.

The flow may be laminar but become turbulent and relaminarize again, and accurate boundary condition assignment is necessary for this analysis, depending on the location of the airways and type of diseases.

Boundary conditions are assigned depending on the severity, type of disease and number of airway generation visible after bifurcation, for example; but not limited to:

A) two-step boundary conditions,

B) boundary conditions using the Lump parameter models (1& 2-parameter, Windkessel 3-parameter model and/or 4-parameter boundary conditions) with or without two-step simulation (to predict boundary conditions using two set of CT-images) are used, the values from pulmonary function test, specifically from reference values but not limited to it, based on sex, age and BMI.

C) generalized mass flow rate ratio at each outlet, based on the area ratio of the outlets and the estimated lobar lung volume of a healthy subject.

These boundary conditions are highly dependent on airway generations visibility on CT images; if generations 1-3^{rd} are visible, patient specific mass flow rate ratio with or without lump parameter models are used, if 3-6^{th} are presented two-step boundary conditions are used with or without lump parameter models, if 6^{th}-10^{th} are visible two-step boundary conditions, if 10^{th}-lower generations are visible uniform lobar pressure or mass flow rate ratio can be assigned.

Visible here refers to image clarity for 3-D model generation.

Boundary conditions are consistence of values of one or more parameters which influence flow, pressure and other parameters in the fluid domain and solid domain, if applicable.

3-D model morphing without structural information (tissue properties) of the airway surfaces and the computational mesh from one phase to another during CFD simulations, and/or average of the CFD results of inspiration and expiration CT phases provide a higher accuracy regarding the characteristic of the flow. Alternately fluid structure interaction (FSI) can be used to capture the movement of the boundary, however FSI is not required for this process and may provide a higher accuracy or additional information.

Reference values from PFTs, predicted and/or reference from normal or predicted values are used as the boundary condition values, but not limited to, prediction of the velocity or mass flow rate for the analysis.

Boundary condition and patient specific three-dimensional structural model generation preferably be during inspiratory-expiratory cycles, or end inspiratory-expiratory cycles.

Another embodiment is the two-step boundary. In this method, first, specific mass flow rates are assigned to each outlet, according to the patient specific lobar volume changes during respiration. Outlet extensions are then added to dissipate the flow structure and using the results from the first step, individual functions for the outlet pressures at each outlet are assigned to make the flow pressure-driven; however, cases which consist of one set of static images can provide similar accuracy when compared with the two-step simulation with few assumptions regarding volume changes based on manual or automatic measurements in static images of the lung lobes. Thus making other mentioned boundary conditions sufficient for these analyses.

The method may include:

a) acquiring image data (Dicom, etc), from medical imaging while subject is awake;

b) may include measurement of lung lobes and upper airways;

c) development of 3-D models from step a);

d) surface repair and adjustment of inlet and outlet;

e) development of computational grid;

f) may include morphing the structural changes to simulate respiration, if applicable;

g) simulating flow behavior using CFD simulation;

h) post-processing and analyzing the data based on biomarkers; and

i) preferably a comparison of the biomarkers to other patients diagnosed with this system, for variability in the analysis.

The present invention concerns the biomarker (Biomarker-1) developed based on inspiratory pressure-expiratory pressure (Pᵢₙ-Pₑₓ) fitted curve. Pₘ-Pₑₓ measures pressure changes before and after flow limiting segment (FLS), constriction, stenosis or suspected obstruction/disease, in this case ECAC, during inspiration and expiration; the line fitted along the slender side of curve and another line perpendicular to the mentioned line could be used as biomarkers for assessing the severity of multiple diseases.

One embodiment of this matter is the angle of the Pᵢₙ-Pₑₓ curve is close to 45° after intervention (or in patients without ECAC) and close to 0° when a more severe case of ECAC disease is present (FIG. 4).

Another embodiment is to fit the curve base on only minimum and maximum values of Pᵢₙ-Pₑₓ curve.

Another embodiment is using CFD-based biomarkers to evaluate FLS based on pressure changes before and after FLS and average velocity at the location with the maximum obstruction to differentiate between EDAC, Crescent type TBM, and saber-sheath type TBM.

A preferred embodiment relates to a method which includes CFD-based biomarkers (Biomarker-2) to diagnose ECAC based on a measurement of the average pressure changes before and after FLS and velocity magnitude at the maximum constriction adjusted by a coefficient based on BMI, age and/or if other respiratory diseases are present.

Another embodiment relates to a method which includes CFD-based biomarkers (similar to Biomarker-2) to diagnose OSA based on a measurement of the average pressure changes (between Nasopharynx and Larynx region) and velocity magnitude at (oropharynx), adjusted by a coefficient based on BMI, age and/or if other respiratory diseases are present.

In another embodiment, a biomarker (Biomarker-3) is used that is similar to coefficient of pressure, which is ΔP/(V̂2) multiplied by a value based on BMI, age and sex.

Another embodiment is using biomarkers 1-3 for monitoring OSA disease.

Another embodiment is using biomarkers 1-3 for diagnoses of OSA disease.

Another embodiment of the present invention relates to a method for assessing obstructive sleep apnea using CFD coupled with medical imaging system, based on fluid characteristics, velocity magnitude and pressures gradients.

Another product of the invention is the ability to locate/identify the optimal location of stent placement. In severe ECAC disease, the insertion of a stent as an intervention can be challenging; the invention assists to determine the most effective location of stent placement before the actual procedure is implemented. This can minimize the probability of an improper stent placement. Improvement assessment of intervention may be done prior to intervention. This may asses the physician and patient cost benefit factors can be determined when selecting a procedure.

### EXAMPLE

The procedure for obtaining the results are as follow: three-dimensional solid models have been generated from the corresponding CT-Scans of a patient with severe EDAC. The model was then imported into CFD software for analyses. Implicit unsteady simulations of airflow with patient specific boundary conditions have been performed, using a K-w turbulence model.

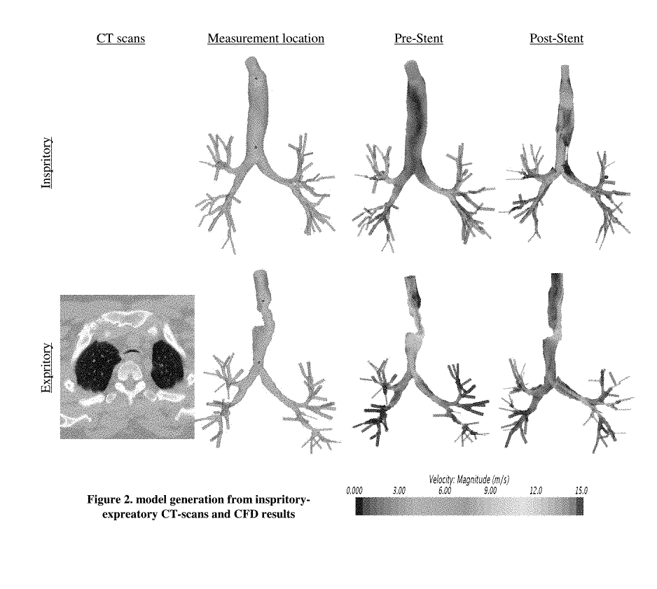

In this patient with sever EDAC the PFTs pre and post stenting (Table 1) show an increase in post stent FEV1 of 290 cc suggesting a decrease in this patients' obstruction and increasing the FEV1/FVC ratio. However, this patient's baseline PFTs are within normal limits confirming PFT's poor sensitivity when evaluating EDAC and/or TBM. The CFD results show pressure drop before (measurement location near inlet) and after flow limiting segment (measurement location downstream obstruction); pre-stent inspiratory-expiratory results show a relative change in pressure of 5.76E-5 cmH2O and 29.5 cmH2O respectively, while inspiration-expiration models for post-stenting have the relative values of 0.0756 cmH2O and 3.14 cmH2O. Flow limiting segments shape (FIG. 2) will have a significant impact on the flow characteristics and can be used as a parameter for development of a biomarker for TBM and EDAC differentiation.

Although the PFT values did not show significant changes for pre and post stenting. Improvement percentage change for pressure drops during exhalation is around 90%, making the CFD based diagnostic and monitoring system a more sensitive tool for evaluation of EDAC compare to PFTs. This system can be used as a monitoring tool for patient's ongoing evaluations.

CFD based diagnostic and monitoring system could be used as a tool with higher sensitivity for evaluation of EDAC compare to PFTs. The slope of the line generated from measuring airway pressure changes proximal and distal to narrow airway during tidal breathing will be used to evaluate ECAC.

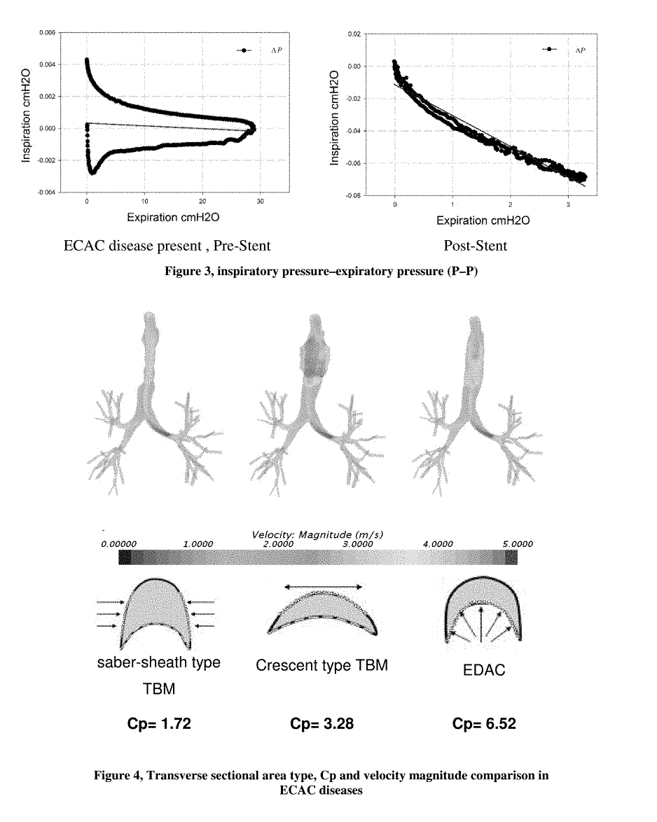

The results show inspiratory pressure-expiratory pressure (P-P) fitted curve will be linear and the angle of the P-P curve is close to 45 degrees after intervention (or in patients without ECAC) and close to 0 degrees when ECAC disease is present (FIG. 3).

The following models were manually adjusted from a patient with EDAC to Crescent type TBM and saber-sheath type TBM to virtually evaluate the flow characteristic, pressure changes (ΔP) immediately upstream and downstream of FLS and their associated Pressure Coefficient (Cp) this is similar but not the same for biomarkers; Cp=ΔP/(0.5ρV̂2), where ρ is the air density and V is velocity (FIG. 4). The reduction of the area increases the airway velocity and an increase in pressure gradient may accentuate the regional collapse. Cp could be used as one of the parameters to differentiate between ECAC diseases.

TABLE 1
PFT results of a patient diagnosed with EDAC before and after Stenting

| Pre-stent Spirometry | | Ref | Pre | % Ref | Post-stent Spirometry | Ref | Pre | % Ref |
|---|---|---|---|---|---|---|---|---|
| FVC | Liters | 4.85 | 4.31 | 89 | FVC | 4.82 | 4.23 | 88 |
| FEV1 | Liters | 3.76 | 3.10 | 82 | FEV1 | 3.73 | 3.39 | 91 |
| FEV1/FVC | % | 78 | 72 | 93 | FEV1/FVC | 77 | 80 | 104 |

We claim:

1. A method for diagnosing and monitoring ECAC consisting steps of:
a. Importing and calculating three-dimensional model of patient with respiratory disease from medical images;
b. Modeling the computational domain using a computer;
c. Assigning boundary conditions based on the airway generations;
d. Modeling by computer the flow behavior and characteristics;
e. Calculating pressure and velocity variations at different airway segments;
f. calculating Flow-based biomarkers base on step d); and
g. Assessing of a patient's condition using flow-based biomarkers.

2. The method of claim 1, further comprising modeling in step d) include computational fluid dynamics, solving Navier-Stokes equations.

3. The method of claim 2, wherein the modeling in step d) incorporates one of K-ω and DES models.

4. The method of claim 1, wherein the assigning of boundary conditions is for air flow modeling.

5. The method of claim 1, wherein results of numerical analysis of step e) is used for biomarker calculation.

6. The method of claim 5, wherein biomarkers are based on pressure values calculated from step e) before and after FLS,

7. The method of claim 5, wherein biomarkers which include flow velocity values calculated from step e) are determined at the maximum airway narrowing.

8. The method of claim 5, where biomarker are demonstrated as single values, graphs, and based on plots fit angle and length of plotted pressure-pressure curves.

9. The method of claim 5, wherein biomarkers are used for OSA disease, which average pressure changes are calculated between Nasopharynx and Larynx region and velocity magnitude at oropharynx.

10. The method of claim 5, wherein biomarkers are used to differentiate between ECAC diseases.

11. A non-transitory computer-readable medium consisting of a computer program with a set of executable instructions that when executed with a computer will perform the following operations:
a. Import medical imaging and calculating three-dimensional model of patient with respiratory disease from medical images;
b. Modeling the computational domain using a computer;
c. Assigning boundary conditions based on the airway generations;
d. Modeling by computer the flow behavior and characteristics;
e. Calculating pressure and velocity variations at different airway segments;
f. Flow-based biomarkers calculation base on step d); and
g. Assessment of patient's condition using flow-based biomarkers.
